(19) United States
(12) Patent Application Publication
(10) Pub. No.: US 2006/0216718 A1
(43) Pub. Date: Sep. 28, 2006

(54) Method and system for estimating gene expression data

(76) Inventors: Antonio Manuel Possolo, Niskayuna, NY (US); Jay Babcock Silkworth, Schodack Landing, NY (US); Kati Illouz, Schenectady, NY (US)

Correspondence Address: Patrick S. Yoder;FLETCHER YODER, P.O. Box 692289, Houston, TX, 77269-2289, US

(21) Appl. No.: 11/087,482

(22) Filed: Mar. 23, 2005

(51) Int. Cl. C12Q 1/68; G06F 19/00

(52) U.S. Cl. 435/6; 702/20

(57) ABSTRACT

A method and system are provided for measuring differential gene expression that is induced on multiple experimental subjects by controlled, multiple experimental factors, measured using microarrays or other devices that measure the activity of multiple probes per gene. The information in the readings made from all the probes in the microarrays or other devices that received the experimental samples is extracted simultaneously, without prior summarization or aggregation at the gene level, by employing a multi-factor model that explicitly accounts for differences in expression between genes, between probes from the same gene, and between the effects of the experimental factors.

### BACKGROUND

The invention relates generally to gene expression analysis and particularly to a method and system for estimating gene expression data in multi-factor microarray experiments.

As a general rule, all cells of a multi-cellular organism, except those cells that are involved in sexual reproduction, whether plant, animal or human, contain a full set of chromosomes with the same set of genes. In any given cell, however, only a fraction of these genes are actively expressed, and these that are so expressed confer to each cell and tissue their unique properties. For example, gene expression typically encompasses the conversion of the information stored in the cell's chromosomes, as a sequence of deoxyribonucleic acid (DNA) base pairs, into a cellular component or product, such as a protein. In particular, the mechanism of gene expression involves the transcription of a subsequence of a DNA molecule pertaining to a gene into a complementary sequence of ribonucleic acid (RNA), typically in the form of a messenger RNA (mRNA) molecule. An mRNA molecule may then be used by the cell as a code that is translated into a protein for use inside or outside the cell. The kind and amount of mRNA produced by a cell or cell type may be studied to learn which genes are expressed by the cell type and under what conditions, which in turn provides insights into how the cell type responds to its changing needs. Such information expands our understanding of the cell's inner workings, and may have biological, toxicological, or medical significance.

One technique by which gene expression may be assessed utilizes microarrays. Microarrays allow the simultaneous study of the expression of thousands of genes under a variety of experimental conditions: therefore, microarrays are particularly useful when one wants to survey a large number of genes. Microarrays may be used to assay gene expression of one particular cell type under uniform conditions, or to measure differential gene expression when the samples of the same cell type or tissue originate from organisms that have been subjected to different experimental conditions: for example, according to whether they did, or did not receive a particular diet supplement, or whether they were, or not, exposed to a particular chemical substance. Statistical techniques, such as regression or analysis of variance (ANOVA), may be used to analyze measurements made using microarrays. For example, such techniques may be used to provide summaries of differential gene expression by comparing the expression of corresponding genes in subjects that have undergone different experimental conditions.

Statistical analysis of microarray data is particularly challenging for several reasons, including, but not restricted to the following. The data sets typically are very large (a single microarray chip may produce more than 200,000 numerical readings, and a typical study may involve tens of such chips, hence millions of numerical values); different probes supposedly measuring the expression of the same gene, may yet produce rather different assessments. Also, replicates of the same biological sample, when applied to different microarrays, may exhibit different responses, owing to various spurious effects (for example, variations in concentration of the reagents that are used and variations in illumination intensity of the microarrays in the process of reading them). Different experimental factors (for example, chemicals administered to the experimental animals the tissue samples are drawn from, or gender of the animals) may interact in non-linear ways, thus adding to the challenge of any statistical analysis of such data. Also, the very nature of the raw data (intensity of fluorescent radiation the samples emit when illuminated with a laser beam, because, as part of the established sample preparation process, they are labeled with a die that fluoresces under such illumination) may require that customized, non-conventional steps be taken prior to their analysis, including the selection and application of non-linear transformations.

Conventional analyses of differential gene expression that use data from microarrays where each gene is represented by multiple probes (each probe is a sub-string of the DNA string that defines the gene's molecular composition), tend to begin by summarizing the readings from the different probes into a single statistical summary (the average, for example), and then carry this summary forward as input to subsequent statistical methods of analysis including analyses of variance, regression analyses, principal components analyses, etc. This discards the variability in the probes' responses, which may be informative in itself, and may dampen the message that the most sensitive probe may convey in each case. In addition, it disregards any non-linear interactions as there may be between probes and experimental treatments.

Conventional experimental techniques also tend to vary one experimental factor at a time, and thus are unable to measure the effects of non-linear interactions between different experimental factors: for example, to measure the effect of being a male and having been exposed to a particular toxin, above and beyond the addition of the separate effects of being male on the one hand, or of having been exposed to the toxin on the other hand.

Therefore, there is a need to provide a technique that is efficient (in the sense that it best extracts all the relevant information in the data), and that can best elucidate the typically complex pattern of relationships involving multiple probes and multiple factors as arise in multi-factor experimentation using microarray platforms where each gene typically is represented by multiple probes.

### BRIEF DESCRIPTION

Briefly in accordance with one aspect, a method for assessing gene expression is provided. The method includes analyzing a set of gene expression data for a plurality of genes acquired by a plurality of probes for each gene and for a plurality of subjects, in such a manner that the gene expression data for all the probes from the same gene is analyzed simultaneously.

In accordance with another aspect, a computer readable media is provided. The computer readable media includes code adapted to analyze a set of gene expression data for a plurality of genes acquired by a plurality of probes for each gene and for a plurality of subjects. The code simultaneously analyzes the gene expression data for all the probes from the same gene.

In accordance with yet another aspect, a system for assessing gene expression is provided. The system includes an interface for receiving gene expression data acquired from different subjects and acquired for multiple genes per subject using multiple probes per gene. The system also includes a processor configured to analyze the gene expression data.

### DRAWINGS

These and other features, aspects, and advantages of the present invention will become clear when the following detailed description is read with reference to the accompanying drawings in which like characters represent like parts throughout the drawings, wherein:

FIG. 1 is a flowchart illustrating exemplary steps for a method for analyzing gene expression data according to aspects of the present technique;

FIG. 2 is a flowchart illustrating exemplary steps for conditioning the gene expression data according to aspects of the present technique;

FIG. 3 is a graphical representation of measurements of differential gene expression of different genes in one particular illustrative example, according to aspects of the present technique; and

FIG. 4 is a diagrammatical representation of an exemplary system for analyzing the gene expression data according to aspects of the present technique.

### DETAILED DESCRIPTION

Aspects of the present technique include a method and system for extracting the information about gene expression that is contained in readings of intensity values from all probes pertaining to each gene. These readings are made on hybridizations involving microarrays, where each gene is represented by multiple probes. The information is extracted from all the probes pertaining to one or multiple genes simultaneously, without prior summarization or aggregation at the gene level, by employing a linear, multi-factor model, in an exemplary embodiment that explicitly accounts for differences in expression between genes, between probes from the same gene, and between the effects of experimental factors (for example, gender of the experimental animals, and toxin the experimental animals were exposed to).

The technique, differently from prior art, preserves the integrity of the possibly discordant readings obtained from probes pertaining to the same gene, and analyses all of them simultaneously, by expressing a suitable function of the probe readings as a linear combination of several factor effects, and of the effects of their interactions. This suitable function of the probe readings that is applied to them in preparation for analysis, comprises correction of the raw readings for background contributions, normalization of inter-array differences that are due to spurious effects, and then logarithmic re-expression.

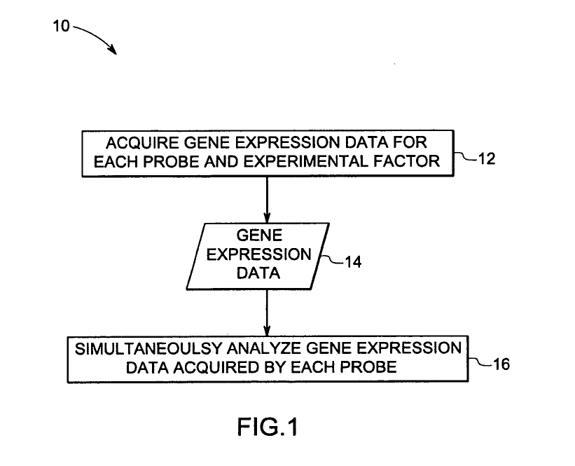

FIG. 1 is a flowchart 10 illustrating exemplary steps of a method for analyzing gene expression data in accordance with the present technique. At step 12, gene expression data 14 is acquired for each gene and experimental subject that had previously been subject to a particular combination of the levels of the experimental factors, by making a sample of genetic material extracted from cells of the experimental subject react with the set of probes pre-assembled in a microarray chip. In one embodiment, the gene expression data 14 is acquired at step 12 by hybridization techniques, such as may be employed for microarrays. For example, each microarray typically has multiple probes for each gene whose expression is measured by the microarray. Different probes transcribe different portions of the same gene, hence may provide different measurements because different portions of the gene may be differently involved in its expression.

In an embodiment employing microarrays to acquire gene expression data 14 at step 12, the data for each probe is typically in the form of a measurement of fluorescent intensity. The data relates to a concentration of the fragments of genetic material in a sample that correspond to the composition of the probes attached to the spot on the microarray where such measurement is made.

Therefore, in such an embodiment, the step 12 of acquiring gene expression data 14 may include placing an exposed microarray into a reader or scanner that may include lasers, a special microscope, and a camera. The laser, microscope and camera work together to create a digital image of the array which contains the intensity values for each probe which are the gene expression data in raw form 14. The gene expression data 14 may be stored in a computer for subsequent analysis. At step 16 the acquired gene expression data 14 are analyzed in such a manner that the information provided by all the probes pertaining to one gene is analyzed simultaneously across all experimental subjects. In one example, the subjects are subjected to different levels of two experimental factors. For example, these factors may include, but are not limited to, gender, foreign chemical substance the subjects have been exposed to, age, diet, environmental conditions, or weight. Furthermore, the gene expression data 14 which is analyzed at step 16 includes the individual probe data without prior summarization or aggregation at the gene level.

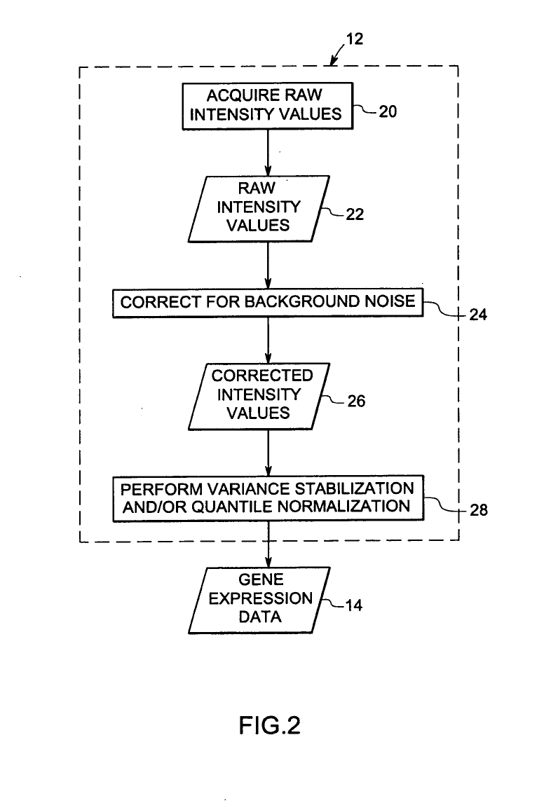

While FIG. 1, provides a generalized overview of the acquisition and analysis of gene expression data 14 in accordance with the present technique, in practice, additional data conditioning steps may be performed in the generation of the gene expression data 14. For example, FIG. 2 depicts a flowchart of steps, some or all of which may be performed as part of the acquisition step 12 in one embodiment. In particular, FIG. 2 depicts steps for conditioning the gene expression data 14 to remove noise from the data.

For example, in this embodiment at step 20 raw numerical intensity values 22 are acquired by reading out the intensity values for each probe of a microarray or other hybridization mechanism. As will be appreciated by those of ordinary skill in the art, these numerical values 22 include not only the expression of the signal emanating from the probe, but also include contributions from possibly several sources of noise that corrupt that signal. Such noise may be referred to as “background noise”. At step 24, such background noise is corrected to condition the data for subsequent analysis. In addition, in the depicted exemplary method at step 28, variance stabilization is performed, by choosing a suitable transformation or re-expression that is applied to the background-corrected measurements of fluorescent intensity. In one example, logarithms of these measurements are taken; this may have the added benefit of improving the linearity of the relationship between these responses and the levels of the experimental factors. In addition, some form of microarray normalization, quantile or other, may be performed to equalize spurious differences between microarrays, as may be due to differences in illumination during measurement, and possibly other causes that are incidental to the experiment. In other exemplary embodiments, one or more of the conditioning steps may be omitted. Further, in other embodiments, other or additional conditioning steps may be performed in generating the gene expression data 14.

Returning to FIG. 1, the gene expression data 14 thus acquired and, typically, background-corrected, transformed, normalized, and/or otherwise conditioned, is analyzed at step 16 without aggregating or summarizing the readings of the multiple probes from each gene, and with simultaneous assessment of the effects of all the experimental factors. For example, a linear, multi-factor model is used in one embodiment to achieve this. The linear, multi-factor model explicitly accounts for differences in expression between genes, between probes from the same gene, and between the effects of experimental factors (which in one embodiment are gender of the experimental animals, and chemical substance they have been exposed to). The effects of the experimental factors on gene expression are estimated simultaneously, rather than one-at-a-time. In this way, the analysis corresponds to the experimental design, where the factor levels are varied together and simultaneously, rather than one at a time, this enabling the assessment of possible, non-linear interactions between experimental factors. In one example, the linear model is of the form
Yᵢⱼₙₖ=γₙ+τₖₙ+σₗₙ+(τσ)ₖₗₙ+επⱼₙ+εᵢⱼₖₗₙ

where the symbols have the following meanings:

| | |
|---|---|
| Yᵢⱼₙₖ | Background corrected, normalized logarithm of the intensity of |
| | the perfect-match probe j of gene n, as observed when the kth |
| | level of the experimental treatment was applied to animal i |
| γₙ | Effect of gene n (average difference to baseline consisting of |
| | expression for control male rats) |
| τₖₙ | Effect of experimental treatment k upon the expression of gene n |
| σₗₙ | Effect of gender of experimental animal upon expression of |
| | gene n |
| (τσ)ₖₗₙ | Non-linear interaction between treatment and gender for gene n |
| πⱼₙ | Effect of probe j from gene n |
| εᵢⱼₖₗₙ | Residual effect that is specific to animal I |

In one embodiment, the model may be fitted either by standard least squares, or by some robust statistical procedure that secures protection against outliers, and that also enhances the boundaries of validity of such statistical inferences as may be derived from the application of the model to the data.

In embodiments employing such a linear, multi-factor model, greater sensitivity to changes in gene expression may be obtained relative to other analysis techniques. This enhanced sensitivity is due to the explicit inclusion of additional factors that may not be of primary interest. For example, inclusion of gender or other factors which are not related to an experimental treatment increases experimental precision because it removes a source of variability that otherwise would inflate the assessment of experimental error. The assessment of experimental error, in turn, provides the baseline against which the statistical significance of the experimental factors (such as toxin and/or drug response) is measured.

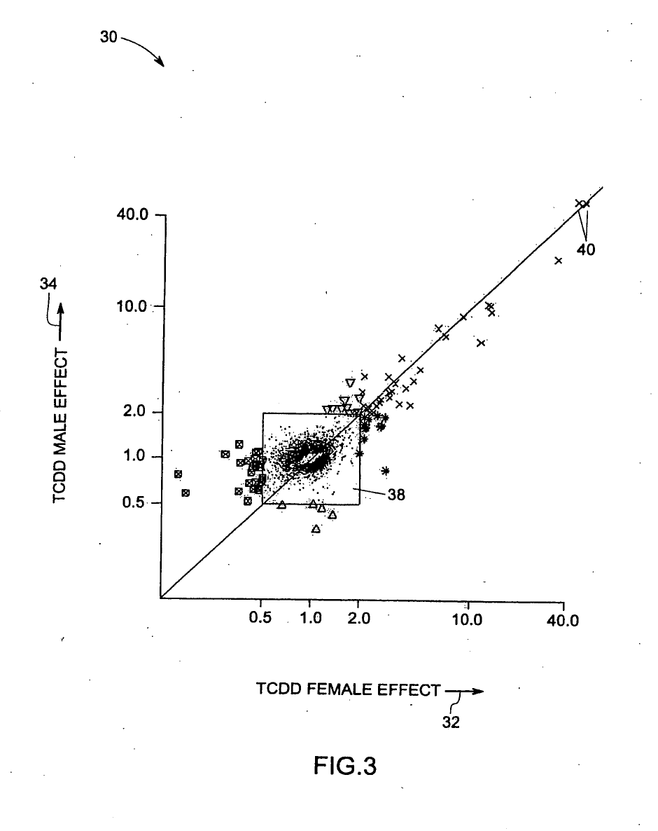

FIG. 3 is a graphical representation of some of the results of an analysis where a linear, multi-factor model was employed to measure differential gene expression data 14 in accordance with the aspects of the present technique. Graph 30 of FIG. 3 depicts the effect of a toxin, TCDD, on gene expression among females (shown on axis designated generally as 32) and males (shown on axis denoted generally by 34). Each point in the graph 30 purports to one gene, and its coordinates are the measures of differential expression of such gene among females (horizontal coordinate 32), and among males (vertical coordinate 34). All of the genes depicted have p-values, associated with TCDD either as main effect or interaction, of less than 0.0001 (p-values measure statistical significance of an effect: the smaller their values, the greater the effect's statistical significance). For those genes whose representing points fall within box 38, although significantly differentially expressed, such differential expression likely is of little biological significance, because it is between 0.5 and 2 fold, meaning that they are either 0.5 to 2 times more expressed, in consequence of the presence of TCDD, than the same genes when the experimental subjects underwent a baseline treatment combination (in the particular embodiment illustrated in this figure, males who received corn oil).

The genes whose differential expression is both statistically and biologically significant are depicted by points outside box 38. For example, the genes denoted by reference number 40 show about 40-fold increase in both males and females. This analysis provides useful conclusions about the effect of a chosen experimental factor and the interaction of different factors (gender and toxicity in this case). This analysis, and these means of summarizing and presenting the results, are useful in identifying groups of genes that tend to behave together in the face of particular treatments or other experimental conditions, and suggest biological pathways that are responsive to such treatments and conditions. The different symbols used to mark the positions of the plotting points in the figure indicate groups of genes whose behavior has some essential, common feature: in this particular embodiment, and for example, genes depicted with multiplication signs (“X”) are up-regulated both in males and females; while those represented by triangles with one vertex pointing up (“A”) (below the square box 38) are down-regulated in males, but do not show biologically significant differential expression among females. The aspects of the technique described herein open new avenues in pharmacological and toxicological studies. The technique may be useful also for tumor classification, risk assessment and prognosis prediction, and for drug development, drug response, therapy development, and tracking disease progression.

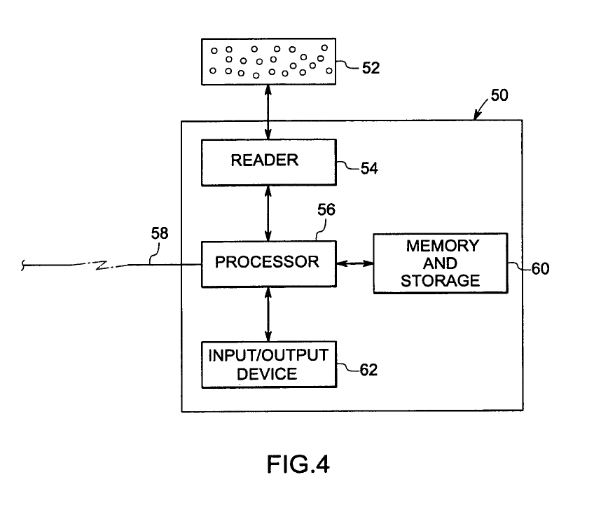

As will be appreciated by those of ordinary skill in the art, the techniques described above with reference to FIGS. 1-3 may be performed on a processor-based system, such as a suitably configured general purpose computer or application specific computer. For example, FIG. 4 is a diagrammatic representation of an exemplary processor-based system 50 for performing the technique as explained with reference to FIGS. 1-2. In one embodiment, the system 50 includes a reader 54 configured to read a microarray 52, as described above. In this embodiment, the reader may include lasers, a special microscope, and a camera. Alternatively, in another embodiment the gene expression data may be provided to the system 50 and the processor 56 not by a microarray reader but by a network or other communication connection 58 configured to access the gene expression data from a remote location, such as a server or other storage device or a remote microarray reader. A memory and storage device 60 may be coupled to the processor 56 for storing the results of the analysis or for storing gene expression data 14 for future analysis. Likewise, routines for performing the techniques described herein may be stored on the memory and storage device 60. The memory and storage device 60 may be integral to the processor 56, or may be partially or completely remote from the processor and may include local, magnetic or optical memory or other computer readable media, including optical disks, hard drives, flash memory storage, and so forth. Moreover, the memory and storage device 60 may be configured to receive raw, partially processed or fully processed data for analysis. An input/output device 62 may be coupled to the processor 56 to display the results of analysis, which may be in the form of graphical illustration as shown in FIG. 3, and/or to provide operator interaction with the processor 56, such as to initiate or configure an analysis. In one embodiment, the input/output device 62 will include one or more of a conventional keyboard, a mouse, a voice recognition routine, or other operator input device. The display/output device will typically include a computer monitor for displaying the operator selections, as well as for viewing the results of analysis according to aspects of the present technique. Such devices may also include printers or other peripherals for reproducing hard copies of the reconstructed images. The processor comprises both hardware and software components: and, among the latter, there will be a customized application that provides a computer implementation of the analytical procedure as described herein in accordance with the aspects of the present technique.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for assessing gene expression, the method comprising:
analyzing a set of gene expression data for a plurality of genes acquired by a plurality of probes for each gene and for a plurality of subjects, wherein the gene expression data for all the probes from the same gene is analyzed simultaneously.

2. The method of claim 1, wherein the plurality of subjects are differentiated by having undergone the same or different levels of at least two experimental factors.

3. The method of claim 2, wherein the at least two experimental factors comprise at least two of gender, toxicity, chemical treatment level, age, disease condition, diet, environmental conditions, activity level, body type, physical condition, weight, body fat, or body muscle.

4. The method of claim 1, wherein the gene expression data is conditioned to remove background noise.

5. The method of claim 1, wherein the gene expression data is conditioned to stabilize variance and to improve the linearity of the response to the effects of the experimental factors.

6. The method of claim 1, wherein the gene expression data is conditioned by application of a normalization technique to the gene expression data.

7. The method of claim 1, further comprising acquiring the set of gene expression data using a microarray having the plurality of probes.

8. The method of claim 1, further comprising applying a multi-factor model for analyzing the gene expression data for all the probes pertaining to each gene simultaneously.

9. A computer readable media, comprising:
code adapted to analyze a set of gene expression data for a plurality of genes acquired by a plurality of probes for each gene and for a plurality of subjects, wherein the gene expression data for all the probes from the same gene is analyzed simultaneously.

10. The computer readable media of claim 9 further comprising code adapted to display a corresponding analysis for the set of gene expression data for the plurality of subjects based on at least two experimental factors.

11. The computer readable media of claim 10, wherein the at least two experimental factors comprise at least two of gender, toxicity, chemical treatment level, age, disease condition, diet, environmental conditions, activity level, body type, physical condition, weight, body fat, or body muscle.

12. The computer readable media of claim 9, wherein the gene expression data is conditioned to remove background noise.

13. The computer readable media of claim 9, wherein the gene expression data is conditioned to stabilize variance and to improve the linearity of the response to the effects of the experimental factors.

14. The computer readable media of claim 9, wherein the gene expression data undergoes application of a normalization technique.

15. The computer readable media of claim 9, further comprising code adapted to apply a multi-factor model for analyzing the gene expression data for all the probes pertaining to each gene simultaneously.

16. A system for assessing gene expression, the system comprising
an interface for receiving gene expression data acquired from a plurality of subjects and acquired for a plurality of genes per subject using a plurality of probes per gene; and
a processor configured to simultaneously analyze the gene expression data.

17. The system of claim 16, wherein the plurality of subjects are differentiated by having undergone the same or different levels of two or more experimental factors.

18. The system of claim 17, wherein the two or more experimental factors comprise at least two of gender, toxicity, chemical treatment level, age, disease condition, diet, environmental conditions, activity level, body type, physical condition, weight, body fat, or body muscle.

19. The system of claim 16, further comprising a microarray coupled to the processor, wherein the microarray comprises the plurality of probes.

20. The system of claim 16, wherein the processor comprises a multi-factor model for analyzing the gene expression data for each probe simultaneously.
